(19) United States
(12) Patent Application Publication
(10) Pub. No.: US 2015/0044725 A1
(43) Pub. Date: Feb. 12, 2015

(54) METHOD OF SEPARATING NUCLEIC ACIDS

(71) Applicant: Samsung Electronics Co., Ltd., Suwon-si (KR)

(72) Inventors: Chang-eun YOO, Seoul (KR); Yeon-jeong Kim, Yongin-si (KR); Dong-hyun Park, Chuncheon-si (KR); Kyung-yeon Han, Seoul (KR)

(73) Assignee: Samsung Electronics Co., Ltd., Suwon-si (KR)

(21) Appl. No.: 14/222,314

(22) Filed: Mar. 21, 2014

(30) Foreign Application Priority Data
Aug. 7, 2013 (KR) 10-2013-0093790

(51) Int. Cl. C12Q 1/68

(52) U.S. Cl. CPC C12Q 1/6806
USPC 435/91.2; 536/25.42; 252/190

(57) ABSTRACT

A method of separating nucleic acids from cells, the method comprising incubating a sample comprising cells with a solid substrate that binds to the cells, whereby the cells adhere to the solid substrate; suspending the solid substrate adhered to the cells in a lysis composition comprising about 100 mM to about 300 mM of alkaline metal salt, and having a pH of about 6 to about 8; lysing the cells in the lysis composition to obtain a lysed solution; and obtaining the nucleic acids from the lysed solution; as well as related compositions and kits.

### CROSS-REFERENCE TO RELATED APPLICATION

This application claims the benefit of Korean Patent Application No. 10-2013-0093790, filed on Aug. 7, 2013 in the Korean Intellectual Property Office, the entire disclosure of which is hereby incorporated by reference.

### INCORPORATION-BY-REFERENCE OF MATERIAL ELECTRONICALLY SUBMITTED

Incorporated by reference in its entirety herein is a computer-readable nucleotide/amino acid sequence listing submitted herewith and identified as follows: 2,136 bytes ASCII (Text) file named “715712_ST25.TXT,” created Mar. 6, 2014.

### BACKGROUND

1. Field

The present invention relates to methods of separating nucleic acids from cells by using compositions including salts, compositions for preventing the adsorption of nucleic acids to surfaces of solid substrates, and kits for separating nucleic acids from cells.

2. Description of the Related Art

Most cancer patients do not die from primary tumors. Rather, these patients usually die from the metastasis of malignant tumor cells that move throughout the body. Metastasis includes a series of complex sequential phases as follows: 1) an expansion stage during which tumor cells expand from primary site to surrounding tissues, 2) an invasion stage during which the tumor cells penetrate into body cavities and blood vessels, 3) an emission stage during which the tumor cells are released through a circulatory system to be transported to distant locations, 4) a re-invasion stage during which the tumor cells re-invade tissues, and 5) an adjustment stage during which the tumor cells adjust to a new environment to facilitate their survival, formation of blood vessels, and growth of tumors.

Biological samples obtained from cancer patients may include rare cells such as circulating tumor cells (CTCs). CTCs include markers that are generally not found in cells of healthy individuals. Accordingly, such markers may be used to separate rare cells such as CTCs from other cell types in biological samples, extract genomic DNAs of the rare cells, and amplify the genomic DNAs to analyze genetic mutations. Through the analysis of the genomic DNA in the rare cells, information useful for the early diagnosis of cancer, prediction of patient survival, and prescription of suitable antitumor agents may be obtained.

Because only very small numbers of CTCs exist in blood, methods of efficiently separating CTCs, and analytical technology for obtaining information from separated CTCs, are needed.

Conventional methods of separating the CTCs in blood include the separation of CTCs using immunoaffinity by fixing antibodies specific for CTCs (e.g., anti-EpCAM) on a solid substrate, (e.g., beads or microchips) followed by extracting the genomic DNA from cells that bind to the solid substrate. In order to minimize loss of genomic DNA, extraction of genomic DNAs from the separated CTCs may require extracting the genomic DNAs without separating the CTCs from the beads. Extracting the genomic DNA without removing the CTCs from the solid substrate however, runs this risk that the genomic DNAs may be adsorbed to the solid substrate, such that the genomic DNA may not be suitable for follow-up analyses (e.g., ligation and RT-qPCR). In the case of conventional methods of extracting genomic DNAs from rare cells such as CTCs, the presence or the absence of a solid substrate is not considered, and thus, adsorption may occur depending on the method.

Accordingly, a need remains for methods and compositions that improve the extraction of DNA from rare cells, such as CTCs, by preventing the adsorption of genomic DNA of cells bound to a solid substrate that has been used to separate the cells.

### SUMMARY

Provided is a method of separating nucleic acids from cells, the method comprising incubating a sample comprising target cells with a solid substrate that binds to the cells, whereby the cells adhere to the solid substrate; suspending the solid substrate adhered to the cells in a lysis composition comprising about 100 mM to about 300 mM of alkaline metal salt, and having a pH of about 6 to about 8; lysing the cells in the lysis composition to obtain a lysed solution; and retrieving the nucleic acids from the lysed solution. Related methods and compositions also are provided.

### BRIEF DESCRIPTION OF THE DRAWINGS

These and/or other aspects will become apparent and more readily appreciated from the following description of the embodiments, taken in conjunction with the accompanying drawings of which:

FIG. 1 is a graph of amplicon concentration plotted against pH illustrating the extent of DNA amplification according to the pH of various suspension solutions;

FIG. 2 is a graph of amplicon concentration plotted against salt concentration illustrating the extent of DNA amplification according to the concentration of salt of various suspension solutions;

FIG. 3 is a graph of amplicon concentration plotted against formamide concentration illustrating the extent of DNA amplification according to the concentration of organic solvent of various suspension solutions; and

FIG. 4 is a graph of amplicon concentration plotted against coating concentration illustrating the extent of DNA amplification according to the coating of various solid substrates.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description.

Provided is a method of separating nucleic acids from cells, the method including: (i) incubating a sample including cells and a solid substrate (ii) adhering the cells onto the solid substrate; (ii) suspending the solid substrate adhered to the cells in a composition including from about 100 mM to about 300 mM of alkaline metal salt, wherein the solution has a pH from about 6 to about 8; (iv) lysing the cells to obtain a solution; and (v) obtaining nucleic acids therefrom.

The method can be used with any cell type; however, the method is believed to be particularly useful for use with rare cells.

The cells, particularly rare cells may exist in a biological sample. The biological sample may comprise saliva, urine, blood, blood serum, bodily tissues, and cell culture medium. Examples of cells include endothelial cells, embryonic cells from embryonic circulation, bacterial cells, cardiac myocytes, epithelial cells, and virus infected cells. The biological sample may be a normal (non-diseased) sample, or an abnormal (diseased) sample.

The rare cells, because they generally do not exist in normal biological samples, may be suitable as markers for abnormal states which may include but are not limited to infectious disease, chronic disease, tissue injuries, or pregnancy. Rare cells include circulating cells metastasized or micro-metastasized from a solid tumor. Circulating cells of a solid tumor include, but are not limited to, circulating tumor cells (CTCs), cancer stem cells, and cells moving to a tumor (e.g., by chemical attraction) such as circulating endothelial precursor cells, circulating endothelial cells, circulating pro-angiogenic myelocytes, and circulating dendritic cells.

CTCs are cells that are detached from tumor cells and circulate in blood vessels, and may be obtained from a biological sample or cell culture fluid obtained from patients with metastatic cancer. Metastatic cancer includes colorectal cancer, small intestine cancer, rectal cancer, anal cancer, esophageal cancer, pancreatic cancer, stomach cancer, kidney cancer, uterine cancer, breast cancer, lung cancer, lymph node cancer, thyroid cancer, prostate cancer, leukemia, skin cancer, colon cancer, brain cancer, bladder cancer, ovarian cancer, and gallbladder cancer.

The solid substrate may comprise any shape. The solid substrate may comprise a spherical, plate, bead, or polygonal shape. The solid substrate may comprise a microchip. For embodiments of the invention having a bead-shaped solid substrate, the size (diameter) of the bead may comprise a size from about 90 nm to about 150 nm, about 5 nm to about 1,000 μm, or about 1 μm to about 50 μm. The bead may comprise a magnetic bead, a silica bead, a polystyrene bead, a glass bead, or a cellulose bead.

For embodiments of the invention comprising a magnetic bead, the magnetic bead may comprise one or more materials selected from the group consisting of rigid metals such as Fe, Ni, Cr, and oxides thereof. The bead may include magnetic silica beads. The bead may comprise a polymer, an organic material, silicon, or glass coated with rigid metal.

Suitable beads comprise commercially available beads such as Dynabeads Genomic DNA Blood (Invitrogen), Dynabeads anti-E. coli O157 (Invitrogen), CELLection™ Biotin Binder Kit (Invitrogen), or MagAttract Virus Min M48 Kit (Qiagen).

The surface of the bead or the microchip may comprise ligands that have affinity for target cells (e.g., rare cells such as CTCs). The ligands may include antibodies, nucleic acids, and proteins. In embodiments of invention where the ligand is an antibody, the antibodies may include one or more types of antibodies that bind specifically to cell determination factors of the target cells, such as rare cells. For example, a bead may be coated with streptavidin to bind antibodies having biotin, forming antibody-bound beads, which in turn may bind cell determination factors of rare cells. When a sample including rare cells is exposed to antibody-bound beads, the antibodies that are capable of specifically binding to specific rare cells may bind to the rare cells. When antibodies on the surface of the magnetic beads and bind to cell determination factors of rare cells, a magnetic field gradient may be used to separate only the beads bound to the rare cells.

The antibodies may comprise antibodies that bind to tumor-associated antigens. The tumor-associated antigens to which the antibodies bind may include EpCAM, tumor-associated glycoprotein-72 (TAG-72), tumor-associated antigen CA 125, prostate specific membrane antigen (PSMA), high molecular weight melanoma-associated antigen (HMW-MAA), Lewis Y tumor-associated antigen, carcinoembryonic antigen (CEA), CEACAM5, HMFG PEM, mucin MUC1, MUC18, and cytokeratin tumor-associated antigen.

The bead bound to the antibody may comprise commercially available beads such as Dynabeads Genomic DNA Blood (Invitrogen), Dynabeads anti-E. coli O157 (Invitrogen), CELLection™ Biotin Binder Kit (Invitrogen), or MagAttract Virus Min M48 Kit (Qiagen).

The solid substrate on which the ligands having a binding capacity to the cells are immobilized, and a sample including the cells, may be incubated. An incubation composition may comprise suitable conditions (e.g., pH, temperature) that promote the binding of ligands coating the solid substrate to target cells.

The solid substrate may be suspended in a composition that includes salt. The salt may be an alkaline metal salt comprising NaCl, KCl, LiCl, or any combination thereof. The addition of salt to a composition may produce effects that prevent the unintended adsorption of nucleic acids from cells bound to the solid substrate. Without wishing to be bound by any particular theory or mechanism of action, it is believed that the strength of attachment of salt ions to the surfaces of particles of the solid substrate is stronger than the strength of adsorption of nucleic acids to the solid substrate, for example, by electrostatic attraction. In other words, the addition of salt may create electrostatic attraction between the surface particles of the solid substrate and the salt sufficient to overcome adsorption of nucleic acids to the solid substrate. The salt may be present at a concentration from about 10 mM to about 400 mM, from about 100 mM to about 300 mM, from about 100 mM to about 400 mM, from about 200 mM to about 300 mM, or about 300 mM.

In some embodiments, the pH of the composition may be from about 6 to about 9, from about 6 to about 8, or about 8. The pH may be adjusted by adding an acidic solution such as HCl and an acetic acid solution, a basic solution such as NaOH, or a suitable buffer solution. When the pH of the composition is within the aforementioned range, synergistic salt-pH effects may be produced that more effectively prevent the adsorption of the nucleic acids to the solid substrate.

The composition may further include an organic solvent. The organic solvent may comprise an aprotic solvent. The aprotic solvent may comprise a solvent without acidic hydrogen, such as acetone, acetonitrile, N, N-dimethyl formamide (DMF), formamide, dimethyl sulfoxide (DMSO), acetamide, or any combination thereof. The organic solvent may be present at a concentration from about 0 volume % to about 10 volume %, e.g., from about 0.5 volume % to about 10 volume %, from about 1 volume % to about 10 volume %, from about 2 volume % to about 10 volume %, from about 5 volume % to about 10 volume %, from about 1 volume % to about 5 volume %, or from about 2 volume % to about 5 volume %. Volume percent compositions refer to the volume of the stated component as a percentage of the total volume of the lysis composition including the cell bound to the solid support.

Lysing of the cells may be performed by sonication, French press cell lysis, homogenization, grinding, freezing-thawing dissolution, and various other methods for physically and mechanically dissolving cells. The nucleic acids may include DNA and RNA.

The expression “obtained solution” as used herein refers to a solution obtained from lysing cells attached to a solid substrate and then removing the solid substrate and cell debris attached to the solid substrate from the lysis solution by using a magnetic substance or a centrifuge. The method according to the present invention substantially prevents the adsorption of nucleic acids, and more particularly genomic DNAs, onto the solid substrate, such that the nucleic acids are included in the obtained solution.

The present invention may further include the step of coating the solid substrate with a zwitterionic material before grinding the cells. The zwitterionic material may include a zwitterionic polymer. The zwitterionic polymer may comprise PCB (polycarboxybetaine) or the like. The zwitterionic material may include 2-methacryloyloxy ethyl phosphorylcholine. The zwitterionic material may be coated at a concentration from about 0% (w/v) to about 0.5% (w/v), from about 0.002% (w/v) to about 0.4% (w/v), or 0.2% (w/v).

The nucleic acids separated by the method described above may be amplified by PCR. The term “PCR” as used herein refers to polymerase chain reaction, wherein polymerase is used to amplify target nucleic acids from primer sets that specifically bind to the target nucleic acids. The PCR method is well known in the art and a commercially usable kit may be used. The PCR method may comprise real-time PCR or reverse-transcription quantitative polymerase chain reaction (RT-qPCR). The term RT-qPCR as used herein is a real-time PCR method in which RNA is amplified using complementary DNA (cDNA) by using reverse transcriptase.

In another embodiment of the invention, the composition for preventing the adsorption of nucleic acids to a solid substrate comprises a pH from about 6 to about 8, and includes from about 100 mM to about 300 mM of alkaline metal salt, wherein the alkaline metal salt is NaCl, KCl, LiCl, or a combination thereof.

The aforementioned composition may further include an organic solvent. The organic solvent may comprise an aprotic solvent such as acetone, acetonitrile, N,N-dimethyl formamide (DMF), formamide, dimethyl sulfoxide (DMSO), and acetamide.

Target cells bound to a solid substrate that are suspended in the aforementioned composition including a salt may prevent the adsorption of the nucleic acids to the solid substrate more effectively than by suspending the target cells and the solid substrate with deionized water.

According to another aspect of the present invention, provided is a kit for separating nucleic acids from cells, wherein the kit includes the composition described above. The kit may include a solid substrate on which ligands having affinity to target cells are immobilized, a lysis solution, and a washing solution. The solid substrate may include beads or microchips.

When extracting nucleic acids, the compositions described above facilitate reduction of the adsorption of exposed nucleic acids to the solid substrate after dissolving cell membranes of the cells that are bound to the solid substrate. As a result, the loss of genomic DNAs from rare cells such as CTCs that exist in small quantities in bodily fluid may be reduced, thereby maximizing the ability to obtain as much information as possible from the genomic DNAs.

Also, when the composition, the kit, or the method of separating the nucleic acids as described above are used, DNAs for the analysis of genetic information of rare cells such as the CTCs that have been separated without the removal of isolates through separate physical manipulation may be efficiently extracted and amplified.

The following examples further illustrate the invention but, of course, should not be construed as in any way limiting its scope.

### Example 1

### A Composition for Preventing the Binding of Nucleic Acids to the Surface of a Solid Substrate

HCC827 (ATCC) cells, which are epithelial cells of human lung tissues, were cultivated in a RPMI1640 culture medium buffered with 10% FBS (Fetal Bovine Serum) and then the cells were retrieved by using Trypsin-EDTA (sigma), followed by the quantification of the cells by using a Sceptor (Millipore), which is a cell quantifying apparatus. Then, serial dilution was used to control the number of target cells such that about 10 cells entered about 2 μl to about 3 μl of the diluted solution, and then the cells were seeded in a 96 well plate to confirm the precise number of cells.

When a desired number of cells were counted, the cells were rotated for 20 minutes with 100 μl of DynaBead® Epithelial Enrich (available from Invitrogen) that had been pre-treated according to a protocol, in a 1.5 ml tube including 1 ml of Phosphate Buffered Saline (PBS). The DynaBead® is a magnetic bead that has been coated with BerEP4 monoclonal antibodies to EpCAM, which is a human epithelial antigen. The DynaBead® has an average diameter of about 4.5 μm. Then, a supernatant was removed by using a magnet.

After suspending the beads bound to the cells prepared as described above in a solution having conditions as described below, nucleic acids were separated and amplified according to a protocol of GenomePlex® Single Cell Whole Genome Amplification Kit (available from Sigma). In greater detail, cell lysis and fragmentation, library preparation, and DNA amplification were processed sequentially without a pause, according to the protocol of GenomePlex® Single Cell Whole Genome Amplification Kit (available from Sigma). In greater detail, the cell lysis and fragmentation were first performed by adding the beads bound to the separated cells in a 9 μl of a solution having the conditions described below and then suspending the same. The suspension was performed by vortexing the tube including the beads and the solution for 10 seconds. As a control, the beads bound to the cells were added to 9 μl of deionized water and then suspended under the same conditions. Then, 2 μl of Proteinase K solution was added to 32 μl of a 10× single cell lysis and fragmentation buffer to prepare a single cell lysis and fragmentation buffer solution. The single cell lysis and fragmentation buffer solution was then completely vortexed to prepare a mixture. 1 μl of the single cell lysis and fragmentation buffer solution was added to the suspension and then completely vortexed. The mixture was incubated at a temperature of 50 degrees Celsius for 1 hour and then heated to a temperature of 99 degrees Celsius, precisely over 4 minutes. The mixture was then cooled with ice and spun down to be used for library preparation.

(1) Extraction and Amplification According to pH

The genomic DNA extraction and amplification experiments according to pH were performed under 4 different conditions shown in Table 1 below. A salt concentration of a suspension composition was maintained at the same level (at 300 mM) and 2-(N-morpholino)ethanesulfonic acid (MES), 1×PBS (150 mM NaCl and 15 mM sodium phosphate), and Tris-EDTA (TE) buffer (10 mM Tris and 1 mM EDTA) were used to adjust pH of the suspension composition to 6.0, 7.4, and 8.0, respectively. Condition 4 is a Comparative Example, which included suspension by using deionized water.

TABLE 1

| | 1 | 2 | 3 | 4 |
|---|---|---|---|---|
| pH | 6.0 | 7.4 | 8.0 | Deionized |
| | | | | water |
| Buffer reagent | MES (10 mM) | 1X PBS | 1X TE | |

| | |
|---|---|
| Final salt (NaCl) | 300 mM |
| concentration | |

FIG. 1 is a graph illustrating the extent of amplification according to the pH of a suspension solution. As shown in Table 1 and FIG. 1, under condition 1 when pH is 6.0, the extent of DNA amplification was about 90 ng/μl, which was lower than the extent of amplification when deionized water was used. Under condition 2 when pH is 7.4 and condition 3 when pH is 8.0 the extent of each DNA amplification was greater than the comparative example. More particularly, the extent of amplification was the greatest under condition 3 when a TE buffer at pH 8.0 was used.

(2) Extraction and Amplification According to Salt Concentrations

Genomic DNA extraction and amplification experiments according to salt concentrations were performed under 4 different conditions shown in Table 2 below. The pH of suspension composition was uniformly maintained at 8.0, and salt concentrations of the suspension composition were adjusted to 150 mM, 300 mM, and 450 mM, respectively. Condition 4 was used as a Comparative Example, which involved suspension by using deionized water.

TABLE 2

| | 1 | 2 | 3 | 4 |
|---|---|---|---|---|
| Final salt (NaCl) | 150 mM | 300 mM | 450 mM | D.W. |

| | |
|---|---|
| concentration | 1X TE (pH 8.0) |
| Buffer reagent | |

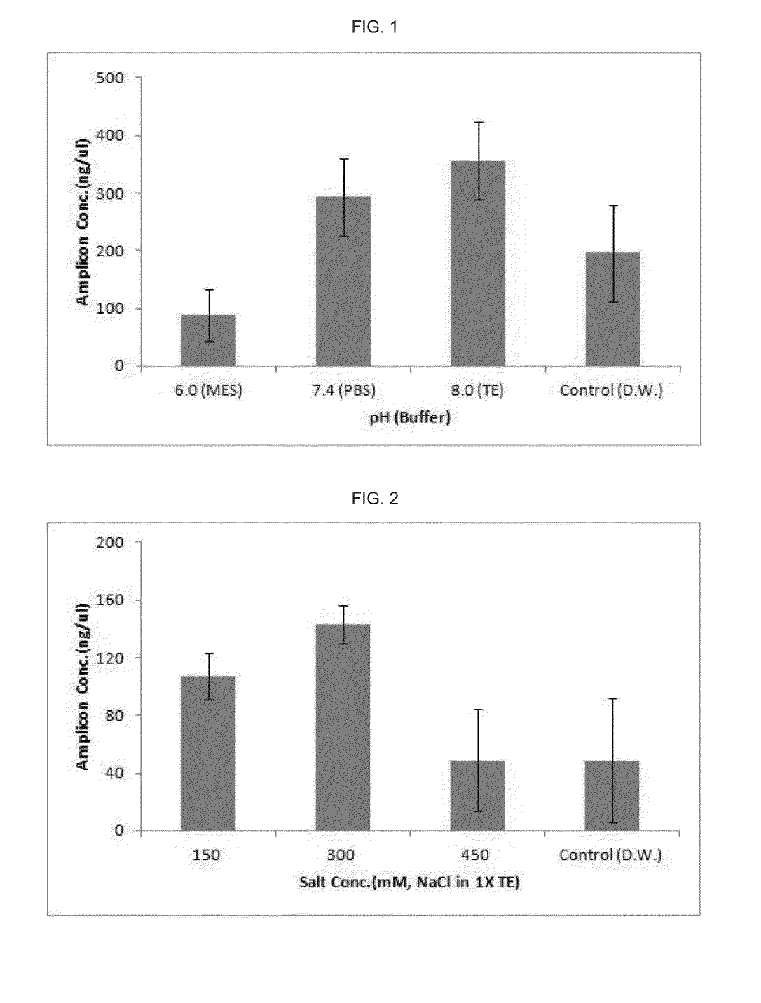

FIG. 2 is a graph illustrating the extent of amplification according to a concentration of salt at a selected pH of a suspension solution. As shown in FIG. 2, when the salt concentration was 150 mM or 300 mM, the extent of amplification was much greater than the Comparative Example in which deionized water was used; however, when the salt concentration was 450 mM, the extent of amplification was comparable to the Comparative Example. More particularly, the extent of amplification was the greatest when the salt concentration was 300 mM.

(3) Extraction and Amplification According to Organic Solvents

Genomic DNA extraction and amplification experiments according to organic solvents were performed under 4 different conditions shown in Table 3 below. The pH and salt concentrations of a suspension composition were uniformly maintained at a pH of 8.0 and NaCl concentration of 300 mM, and formamide content were adjusted to 0%, 5%, and 10%, respectively. Condition 4 was used as a Comparative Example, which involved suspension by using deionized water.

TABLE 3

| | 1 | 2 | 3 | 4 |
|---|---|---|---|---|
| Formamide (%) | 0 | 5 | 10 | D.W. |

| | |
|---|---|
| Buffer reagent | 1X TE |
| | (pH 8.0) |
| Final salt (NaCl) | 300 mM |
| concentration | |

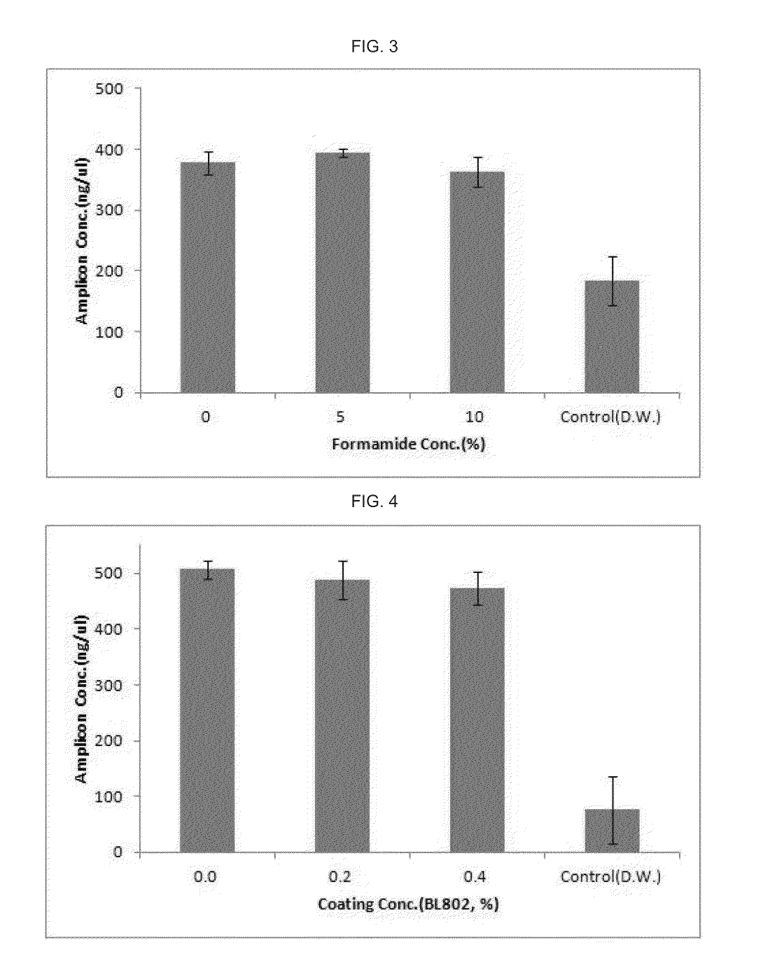

FIG. 3 is a graph illustrating the extent of amplification according to a concentration of an organic solvent at a selected pH of a suspension solution and a selected concentration of salt. As shown in FIG. 3, there was a small increase in the amplification of DNA when organic solvent was added. The increase was comparatively the greatest at 5%.

(4) Extraction and Amplification According to Polymer Coating

After incubating cells and beads to bind the cells and the beads, the beads bound to the cells were added to 500 μl of 0%, 0.2%, or 0.4% 2-methacryloyloxy ethyl phosphorylcholine (BL802, available from BioLipidure) solution to prepare a mixture, the mixture was rotated for 15 minutes, and the rotated mixture thereof was washed once with PBS to completely remove a supernatant. Each of the prepared beads was added to 9 μl of a 1× Tris-EDTA (TE) buffer solution containing 5% formamide and 300 mM of NaCl, and the resultant mixture thereof was suspended. Then, nucleic acids were separated and amplified from the suspension solution according to GenomePlex® Single Cell Whole Genome Amplification Kit (available from Sigma) protocol. As a control, the beads were not coated and the beads bound to the cells were suspended in deionized water.

(5) Quantitative PCR

50 ng of DNA treated and amplified by each condition of Table 4 below, 250 nM of primer, 2×SYBR Master mix (available from Exiqon), and deionized water were mixed to perform quantitative PCR as described below by using LightCycler® C480, which is a PCR apparatus.

In greater detail, a suspension composition having the conditions shown in Table 4 below was prepared. Conditions 1 and 2 are conditions according to the present invention, condition 3 is a comparative condition in which the suspension solution is deionized water (D.W.) and includes beads, and condition 4 is a comparative condition in which the beads are absent and the suspension solution is D.W.

TABLE 4

| | 1 | 2 | 3 | 4 |
|---|---|---|---|---|
| Buffer | 1X TE (pH 8.0) | 1X TE (pH 8.0) | D.W. | D.W. |
| Final salt (NaCl) | NaCl 300 mM | NaCl 300 mM | X | X |
| concentration | | | | |
| Formamide | 5% | 5% | X | X |
| BL 802 coating | X | 0.2% | X | X |
| Beads | ◯ | ◯ | ◯ | X |

Target sequences and primer sequences used in PCR are as shown in Table 5 below. With the target sequences and the primers shown in Table 5, each target sequence was subjected to quantitative PCR by using the primer. With respect to target sequences Ch2, Ch4, Ch12, and Ch13, the target sequences were subjected to one cycle at a temperature of 95 degrees Celsius for 10 minutes, and then 45 cycles at 90 degrees Celsius for 10 seconds, at 60 degrees Celsius for 45 seconds, and at 72 degrees Celsius for 15 seconds. With respect to the target sequence epithelial growth factor receptor 19 (EGFR19), one cycle was processed at 95 degrees Celsius for 10 minutes, and 45 cycles were processed at 95 degrees Celsius for 10 seconds and at 72 degrees Celsius for 15 seconds.

TABLE 5

| Target sequence | |
|---|---|
| (locus) | Primer |
| Ch2 | Forward: SEQ ID NO: 1 |
| | Reverse: SEQ ID NO: 2 |
| Ch4 | Forward: SEQ ID NO: 3 |
| | Reverse: SEQ ID NO: 4 |
| Ch12 | Forward: SEQ ID NO: 5 |
| | Reverse: SEQ ID NO: 6 |
| Ch13 | Forward: SEQ ID NO: 7 |
| | Reverse: SEQ ID NO: 8 |
| EGFR19 | Forward: SEQ ID NO: 9 |
| | Reverse: SEQ ID NO: 10 |

In Table 4, the DNA amplified under conditions 1 to 3 (0, 0.2%, and control) in which the beads are present and condition 4 in which the beads are absent were used to perform quantitative PCR with respect to 5 sites of the genomic DNAs. The results are as follows. Here, a value for each target sequence represents an average of crossing point (Cp) values (standard deviation) (n=6), wherein a Cp value is a periodicity of a point at which an amount of fluorescence generated by separation of probes increases beyond a baseline level, such that the fluorescence can be seen, and the Cp value may be used for quantifying the DNA. Detection % refers to the number of experiments that show noticeable differences from the NTC (no template control), among the total number of experiments (5 sites×6 repetitions).

TABLE 6

| Experiment | | | | | | Detection |
|---|---|---|---|---|---|---|
| conditions | Ch2 | Ch4 | Ch12 | Ch13 | EGFR19 | % |
| 1(0.0%) | 37.1(±1.5) | 23.8(±1.3) | 28.7(±4.7) | 31.9(±4.8) | 23.4(±0.6) | 93 |
| 2(0.2%) | 34.8(±4.2) | 26.4(±5.9) | 34.5(±8.3) | 26.6(±2.4) | 23.9(±0.9) | 87 |
| 3(D.W.) | N.D. | N.D. | N.D. | N.D. | 37.7(±2.0) | 0 |
| 4(No Bead) | 28.0(±6.3) | 22.9(±0.9) | 26.9(±2.1) | 27.6(±6.6) | 23.6(±1.5) | 100 |
| NTC | N.D. | N.D. | N.D. | N.D. | 31.9(±0.2) | |

NTC: no template control
ND: not determined

In Table 6 above, based on the fact that condition 3 was not different from the NTC, it may be concluded that the DNA was not detected. On the other hand, based on the fact that conditions 1 and 2 showed noticeable differences from NTC, it may be concluded that the DNA was detected and there does not seem to be any substantial difference between conditions 1 and 2. Although the Cp value tends to increase in conditions 1 and 2 compared to the case in which the beads are absent, the Cp value is noticeably different from the NTC. Thus, conditions 1 and 2 will not cause problems in DNA detection.

It should be understood that the exemplary embodiments described herein should be considered in a descriptive sense only and not for purposes of limitation. Descriptions of features or aspects within each embodiment should typically be considered as available for other similar features or aspects in other embodiments.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of separating nucleic acids from cells, the method comprising:
incubating a sample comprising cells with a solid substrate that binds to the cells, whereby the cells adhere to the solid substrate;
suspending the solid substrate adhered to the cells in a lysis composition comprising about 100 mM to about 300 mM of alkaline metal salt, and having a pH of about 6 to about 8;
lysing the cells in the lysis composition to obtain a lysed solution; and
obtaining the nucleic acids from the lysed solution.

2. The method of claim 1, wherein the solid substrate further comprises ligands on the surface of the solid substrate that bind to the cells.

3. The method of claim 1, wherein the alkaline metal salt is NaCl, KCl, LiCl, or any combination thereof.

4. The method of claim 1, further comprising coating the solid substrate with a polymer before lysing the cells.

5. The method of claim 4, wherein the polymer is a zwitterionic polymer.

6. The method of claim 1, wherein the composition further comprises an organic solvent.

7. The method of claim 6, wherein the organic solvent is an aprotic solvent.

8. The method of claim 7, wherein the aproptic solvent comprises acetone, acetonitrile, N, N-dimethyl formamide (DMF), formamide, dimethyl sulfoxide (DMSO), acetamide, or any combination thereof.

9. The method of claim 1, further comprising an organic solvent in an amount from about 0 volume % to about 10 volume %.

10. The method of claim 1, further comprising amplifying the nucleic acids obtained from the lysed solution by PCR.

11. A composition comprising about 100 mM to about 300 mM of alkaline metal salt, wherein the alkaline metal salt is NaCl, KCl, LiCl, or a combination thereof, and a pH of about 6 to about 8, and a solid substrate comprising ligands that specifically bind to a cell.

12. The composition of claim 11, further comprising an organic solvent.


SEQUENCE LISTING (10 sequences)

SEQ ID NO: 1 - DNA, 20 bp, Artificial Sequence
  Synthetic (primer)
catggctcactggcttacaa

SEQ ID NO: 2 - DNA, 21 bp, Artificial Sequence
  Synthetic (primer)
ttgcctcttacagaggagcag

SEQ ID NO: 3 - DNA, 22 bp, Artificial Sequence
  Synthetic (primer)
gcaaaatccataccctttctgc

SEQ ID NO: 4 - DNA, 25 bp, Artificial Sequence
  Synthetic (primer)
tctttccctctacaaccctctaacc

SEQ ID NO: 5 - DNA, 23 bp, Artificial Sequence
  Synthetic (primer)
tttgatgttaggacacgctgaaa

SEQ ID NO: 6 - DNA, 23 bp, Artificial Sequence
  Synthetic (primer)
aaaaacggaagaagtctcttggc

SEQ ID NO: 7 - DNA, 23 bp, Artificial Sequence
  Synthetic (primer)
gtcagaagactgaaaacgaagcc

SEQ ID NO: 8 - DNA, 23 bp, Artificial Sequence
  Synthetic (primer)
gcttgccacactcttcttcaagt

SEQ ID NO: 9 - DNA, 20 bp, Artificial Sequence
  Synthetic (primer)
agccaggaacgtactggtga

SEQ ID NO: 10 - DNA, 20 bp, Artificial Sequence
  Synthetic (primer)
ctcactttgcctccttctgc